(19) United States
(12) Patent Application Publication
(10) Pub. No.: US 2007/0117833 A1
(43) Pub. Date: May 24, 2007

(54) Pharmaceutical compositions

(76) Inventor: Axel Maibücher, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/581,069

(22) PCT Filed: Nov. 30, 2004
(86) PCT No.: PCT/EP04/13587

§ 371 Date: May 31, 2006

(30) Foreign Application Priority Data
Dec. 1, 2003 (GB) 0327840.5

(51) Int. Cl. A61K 31/4745

(52) U.S. Cl. 514/291

(57) ABSTRACT

Pharmaceutical combinations comprising at least one mTOR inhibitor and their uses in treating arthritis or rheumatic arthritis and disorders associated therewith.

The present invention relates to pharmaceutical combinations comprising at least a mTOR inhibiting agent, e.g. rapamycin or a rapamycin derivative, and their uses in treating arthritis or rheumatic arthritis and disorders associated therewith.

Rheumatoid arthritis (RA) affects between 1 and 2% of the population and is a progressive illness that has the potential to cause joint destruction and functional disability. It is characterized by hyperproliferation of the synovial membrane with subsequent formation of fibrous pannus which invades and erodes cartilage and bone.

Accordingly, there is a need for agents which are effective in the treatment of arthritis or rheumatic arthritis, e.g. including reduction of, alleviation of, stabilization of or relief from the symptoms or illness which affect the organism, particularly joints or vertebrae, including also slowing the progression (destruction of the joints) in moderate to severe rheumatoid arthritis. progressive, or erosive rheumatoid arthritis who had an inadequate response to treatment with disease-modifying antirheumatic drugs. A further need is the reduction of the side-effects.

It has now been found that a combination comprising at least a mTOR inhibiting agent and a co-agent, e.g. as defined below, has a beneficial effect on arthritis or rheumatic arthritis and the disorders associated therewith, e.g. reducing the signs and symptoms of arthritis or rheumatic arthritis.

In accordance with the particular findings of the present invention, there is provided

1. A pharmaceutical combination comprising:
a) mTOR inhibitor, and
b) at least one co-agent shown to have clinical activity against arthritis or rheumatic arthritis, e.g.RA.

2. 1 A method for treating arthritis, rheumatic arthritis or disorders associated therewith in a subject in need thereof comprising co-administration to said subject, e.g. concomitantly or in sequence, of a therapeutically effective amount of a mTOR inhibitor, e.g. rapamycin or a derivative thereof, e.g. as defined hereinafter, and at least one co-agent, e.g. as indicated hereinafter.

Examples of arthritis and rheumatic arthritis are e.g. RA, arthritis chronica progrediente, arthritis deformans, psoriatic arthritis, polyarthritis, ankylosing spondylitis, polychondritis or osteoarthritis. Disorders associated with such diseases include e.g. pain, pyresis, macrophages or synovial fibroblasts proliferation or bulk formation of invasive fibrous pannus.

2.2 A method for slowing the progression, e.g destruction of the joints, in a subject having moderate to severe rheumatoid arthritis, comprising co-administration to said subject, e.g. concomitantly or in sequence, of a therapeutically effective amount of a mTOR inhibitor, e.g. rapamycin or a derivative thereof, e.g. as defined hereinafter, and at least one co-agent, e.g. as indicated hereinafter.

Accordingly the present invention also provides:

2.3 A method for reducing or inhibiting macrophages or synovial fibroblasts proliferation in a subject in need thereof comprising administration to said subject of a therapeutically effective amount of a mTOR inhibitor, e.g. rapamycin or a derivative thereof, e.g. as defined hereinafter, optionally in combination with, e.g. concomitantly or in sequence, with a therapeutically effective amount of at least one co-agent, e.g. as indicated hereinafter.

2.4 A method for reducing or inhibiting bulk formation of invasive fibrous pannus in a subject in need thereof comprising administration to said subject of a therapeutically effective amount of a mTOR inhibitor, e.g. rapamycin or a derivative thereof, e.g. as defined hereinafter, optionally in combination with, e.g. concomitantly or in sequence, with a therapeutically effective amount of at least one co-agent, e.g. as indicated hereinafter.

2.5 A method for preventing, alleviating or treating pain, e.g. associated with arthritis or rheumatic arthritis diseases, in a subject in need thereof comprising administration to said subject of a therapeutically effective amount of a mTOR inhibitor, e.g. rapamycin or a derivative thereof, e.g. as defined hereinafter, optionally in combination with, e.g. concomitantly or in sequence, with a therapeutically effective amount of at least one co-agent, e.g. as indicated hereinafter.

2.6 A method for preventing, alleviating or treating pyresis, e.g. associated with arthritis or rheumatic arthritis diseases, in a subject in need thereof comprising administration to said subject of a therapeutically effective amount of a mTOR inhibitor, e.g. rapamycin or a derivative thereof, e.g. as defined hereinafter, optionally in combination with, e.g. concomitantly or in sequence, with a therapeutically effective amount of at least one co-agent, e.g. as indicated hereinafter.

3. A pharmaceutical combination as disclosed herein for use in any one of the methods 2.1 to 2.6.

4.1 A pharmaceutical composition for use in any one of the methods 2.1 to 2.6 comprising a mTOR inhibitor, e.g. rapamycin or a derivative thereof, e.g. as defined hereinafter, together with one or more pharmaceutically acceptable diluents or carriers therefor.

4.2 A mTOR inhibitor, e.g. rapamycin or a derivative thereof, e.g. as defined hereinafter, e.g. a Compound of formula 1, for use in any one of the methods 2.1 to 2.6.

4.3 A mTOR inhibitor, e.g. rapamycin or a derivative thereof, e.g. as defined herein after, e.g. a Compound of formula 1, for use in the preparation of a medicament for use in any one of the methods 2.1 to 2.6.

The term “pharmaceutical combination” as used herein means a product that results from the mixing or combining of more than one active ingredient and includes both fixed and non-fixed combinations of the active ingredients. The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “fixed combination” as that term is used herein means that the active ingredients, e.g. the mTOR inhibitor and a co-agent, are both administered to a patient simultaneously in the form of a single entity or dosage.

The term “non-fixed combination” as that term is used herein means that the active ingredients, e.g. the mTOR inhibitor and a co-agent, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

A mTOR inhibitor is a compound which targets intracellular mTOR (“mammalian Target Of Rapamycin”). mTOR is a family member of phosphatidylinositol 3-kinase (PI3-kinase) related kinase. Rapamycin and rapamycin derivatives inhibit the mTOR pathway via a complex with its intracellular receptor FKBP12 (FK506-binding protein 12).

Rapamycin is a known macrolide antibiotic produced by Streptomyces hygroscopicus. By rapamycin derivative is meant a substituted rapamycin having mTOR inhibiting properties, e.g. rapamycin substituted in position 40 and/or 16 and/or 32, for example a compound of formula I

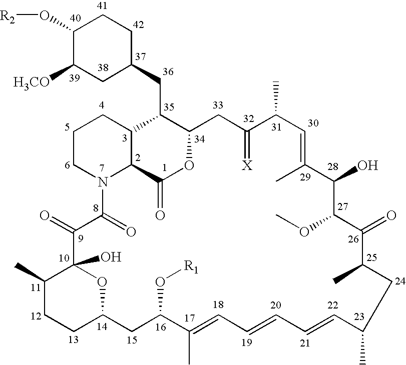

wherein

R₁ is CH₃ or C₃₋₆alkynyl,

R₂ is H, —CH₂—CH₂—OH, 3-hydroxy-2-(hydroxymethyl)-2-methyl-propanoyl or tetrazolyl, and

X is ═O, (H,H) or (H,OH)

provided that R₂ is other than H when X is ═O and R₁ is CH₃,

or a prodrug thereof when R₂ is —CH₂—CH₂—OH, e.g. a physiologically hydrolysable ether thereof, for instance —CH₂—CH₂—O—C₁₋₈alkyl.

Representative rapamycin derivatives of formula I are e.g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxy-methyl)-2-methylpropanoate]-rapamycin (also called CC1779) or 40-epi-(tetrazolyl)-rapamycin (also called ABT578). A preferred compound is e.g. 40-0-(2-hydroxyethyl)-rapamycin disclosed in Example 8 in WO 94/09010 (referred hereinafter as Compound A), or 32-deoxorapamycin or 16-pent-2-ynyloxy-32(S)-dihydro-rapamycin as disclosed in WO 96/41807.

Rapamycin derivatives may also include the so-called rapalogs, e.g. as disclosed in WO 98/02441, WO01/14387 and WO 03/64383, e.g. AP23573, AP23464, AP23675 or AP23841.

Further examples of a rapamycin derivative are those disclosed under the name TAFA-93, biolimus-7 or biolimus-9.

The co-agent b) for use according to the invention may be selected from the following groups of compounds:

i) an antimetabolite, e.g. methotrexate;

ii) a TNFα-inhibitor; e.g. a biological molecule, for instance produced by recombinant DNA technology, e.g. an antibody against TNF-α, e.g. a human monoclonal antibody such as adalimumab (Humira™), a chimeric (mouse and human) monoclonal antibody such as infliximab (Remicade™), a fusion protein comprising a ligand binding portion of the TNFreceptor, e.g. Etanercept (Enbrel™) which is a dimeric fusion protein of the ligand-binding region of the 75-kd (p75) TNF receptor linked to the Fc portion of human IgG1, an antisense oligonucleotide, e.g. ISIS 104838; or a low molecular weight compound, e.g. a pyridinylamide, e.g. JM34 [N-(4,6-dimethylpyridin-2-yl)-furane-2-carboxamide] or JM42 [N-(4,6-dimethylpyridin-2-yl)5-bromofurane-2-carboxamide];

iii) an interleukin antagonist, e.g. an IL-1R inhibitor, e.g. anakinra (Kineret™), an IL-6R inhibitor, e.g. an anti IL-6R monoclonal antibody, e.g. an humanized monoclonal antibody such as atlizumab (Chugai MRA);

iv) a p38 MAP kinase inhibitor, e.g. a pyridinylimidazole compound, for example SB 203580; a quinolin-2-one or isoxazolo [3,4-c]quinolin-2-one, e.g. ICX 56238890 or ICX 56319223 (3-[3-(4-chlorophenyl)-3-naphthalen-2-ylamino)-propanoyl]-4-hydroxy-1-methylquinolin-2(1 H)-one); SCIO-323, SCIO-469; VX-702;

v) a cyclooxygenase inhibitor, e.g. celecoxib (Celebrex™), rofecoxib (Vioxx™), etoricoxib, valdecoxib or a 5-alkyl-2-arylaminophenylacetic acid, e.g. lumiracoxib (Prexige™);

vi) a sulfonamide compound useful in RA, e.g. sulfasalazine (5-(Ip-(2-pyridylsulfamoyl)phenylazo)salicylic acid);

vii)an antimalarial compound, e.g.hydroxychloroquinine or chloroquinine;

viii)an analgesic, e.g. salicylic acid or a derivative thereof, for ex. acetyl salicylic acid, or a benzeneacetic acid derivative, e.g. ibufenac, ibuprofen or ibuproxam.

In each case where citations of patent applications or scientific publications are given, the subject-matter relating to the compounds is hereby incorporated into the present application by reference. Comprised are likewise the pharmaceutically acceptable salts thereof, the corresponding racemates, diastereoisomers, enantiomers, tautomers as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e. a pharmaceutical combination within the scope of this invention could include three active ingredients or more. Further both the first agent and the co-agent are not the identical ingredient.

Utility of the mTOR inhibitors and their combinations in treating arthritis or rheumatic arthritis diseases as hereinabove specified, may be demonstrated in animal test methods as well as in clinic, for example in accordance with the methods hereinafter described.

A.1 Effect on the spontaneous proliferation of human rheumatoid synovial fibroblast Synovial cells obtained by collagenase digestion of synovial tissue from RA patients are dissociated with trypsin/EDTA and cultured in gelatin-coated Petri dishes as suspension cultures in RPMI 1640 medium supplemented with 10% fetal calf serum, 2 mM L-glutamine, 50 U/ml penicillin-50 mg/ml streptomycin (all from Gibco), and 10 mM HEPES. Synovial cells are used at passage 2 through 8 for experiments. Assessment of cell growth is made using either direct detection of DNA synthesis in cells as determined by bromodeoxyuridine incorporation using an assay kit obtained from Amersham or by direct counting of viable cells at the end of the incubation period. Cells are initially seeded at 2×10⁵ cells per well. Statistical analysis is by Student's test. The mTOR inhibitor, e.g. a compound of formula I, e.g. Compound A, significantly suppresses the growth of synovial cells by both criteria. In the case of the bromodeoxyuridine method, maximal effects are observed at 10 pM concentrations. Direct cell counting shows maximal effects when the concentration reaches 10 nM. Studies with rapamycin show that when synovial cell growth is assessed by measuring total intracellular ATP levels, suppression is maximal at 10 pM.

A.2 Antipyretic Effects

LPS fever. An injection of lypopolysaccharide (LPS) at a dose of 100 μg/kg in 5 ml/kg is given subcutaneously, and 2 hours later the temperature is measured using a rectal thermocouple. The rats are then placed into matched treatment groups according to their temperature responses. At time +4 hours, the mTOR inhibitor is administered p.o., and the final temperatures measured again at time +6 hours. The increase in temperature is calculated for each animal and the % inhibition determined for each treatment group compared to the vehicle control group.

IL-1 fever. Baseline temperature is measured and the rats placed into matched groups. The animals are dosed with the mTOR inhibitor (0.5, 2 or 4 mg/kg) p.o., and 30 minutes later given an injection of 100 ng IL-1β i.v. At time +4hours the final temperatures are measured and the % inhibition calculated as for LPS fever.

In these assays, the mTOR inhibitor inhibits LPS and IL-1β induced fever. Compound A shows dose-related inhibition of both LPS und IL-1β-induced fever in rats with ED₅₀'s of 1.9 (1.21-2.41)_{95%} and <0.54 mg/kg p.o., respectively.

A.3 Anti-nociceptive Activity in a Model of Inflammatory Pain

Hyperalgesia is induced by an intra-plantar yeast injection and nociception measured by applying increasing pressure to the foot until the animal vocalizes or withdraws its foot from the pressure pad. The baseline pressure required to induce vocalization or withdrawal of the paw of male OFA rats is measured (−2 hours), followed by an intra-plantar injection of 100 μl of a 20% yeast suspension in water in the hind paw. The rats are treated orally with rapamycin or a derivative thereof (0.5, 2 or 4 mg/kg) or vehicle (saline) p.o. 2 hours later (0 hours), and the pressure test repeated 1 and 2 hours after dosing. The pressure required to induce vocalization or paw withdrawal of the compound-treated rats at these time-points is compared to that of vehicle-treated animals.

In these assay, the mTOR inhibitor inhibits paw hyperalgesia. Compound A significantly inhibits paw hyperalgesia after 1 hour with the 2 mg/kg dose and at both 1 and 2 hours with the 4 mg/kg p.o. dose.

B Clinical Trial

Suitable clinical studies are, for example, open label non-randomized, dose escalation studies in patients with rheumatoid arthritis. Such studies may prove e.g. the synergism of the active ingredients of the combination of the invention. The beneficial effects on arthritic diseases can be determined directly through the results of these studies or by changes in the study design which are known as such to a person skilled in the art. Such studies are, in particular, suitable to compare the effects of a monotherapy using the active ingredients and a combination of the invention. Preferably, the dose of mTOR inhibitor (a) is escalated until the Maximum Tolerated Dosage is reached, and the co-agent (b) is administered with a fixed dose. Alternatively, the agent (a) is administered in a fixed dose and the dose of co-agent (b) is escalated. Each patient receives doses of the mTOR inhibitor (a) either daily or intermittent.

The efficacy of the treatment can be determined in such studies, e.g., after 12, 18 or 24 weeks by evaluation of tender joint count and swollen joint count.

Alternatively, a placebo-controlled, double blind study can be used in order to prove the benefits of the combination of the invention mentioned herein.

120 patients who are methotrexate partial responders are randomized in 2 groups to receive the mTOR inhibitor a) or placebo once daily for 12 weeks while continuing their background methotrexate treatment. There is an initial Screening visit (days −21 to −7) prior to the baseline. Disease status is assessed at the Screening and Baseline visits. During the 12 weeks treatment period the patients will be seen at Weeks 1, 2, 4, 8 and 12 and in the follow-up period at Weeks 14, 16, 20 and 24.

The criteria for inclusion of the patients in the trial: they must have a diagnosis of RA as defined by the 1988 revised ARA criteria with disease duration of no less than 6 months. All patients must have active RA as defined by the following parameters:
at least 6 swollen and 9 tender joints based on 58/60 joint count upon entry into the study
one of the following: erythrocyte sedimentation rate (ESR) >28 mm/hr, C-reactive protein (CRP)>1.5 mg/dL or morning stiffness>45 minutes

Patients must have received methotrexate for at least 16 weeks and must be on a stable dose (≧7.5 mg/week) and route of administration for at least 8 weeks prior to Day 1. They continue to receive the same daily dose of methotrexate during the course of the 12-week treatment.

The primary efficacy outcome measure is the attainment of ACR20 criteria for improvement of RA and the proportion of patients in each group meeting the ACR20 criteria is determined. The ACR20 criteria defines clinical response as 20% improvement in both the tender joint count and the swollen joint count, in addition to 20% improvement in at least three of five variables (degree of disability HAQᵢ, patient global assessment, physician global assessment, pain and CRP/ESR levels).

The mTOR inhibitor, e.g. Compound A, administered, e.g. at a dose of 6 mg/day, in combination with methotrexate leads to a response according to ACR20 superior compared to the placebo group. For example, the results obtained with Compound A are as follows:

ACR Responders (in %) to ACR20

| Time | Compound A | Placebo |
|---|---|---|
| Week 2 | 14.8 | 11.7 |
| Week 12 | 36.1 | 16.7 |

The administration of a pharmaceutical combination of the invention results in a beneficial effect, e.g. a synergistic therapeutic effect, e.g. with regard to alleviating, delaying progression of or inhibiting the symptoms, and/or for effects such as e.g. an improved quality of life or a decreased morbidity, compared with a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, which may diminish the incidence or severity of side-effects. This is in accordance with the desires and requirements of the patients to be treated.

It is one objective of this invention to provide a pharmaceutical composition comprising a quantity, which is jointly therapeutically effective against arthritis, rheumatic arthritis or disorders associated therewith comprising a combination of the invention. In this composition, the mTOR inhibitor a) and co-agent (b) may be administered together, one after the other or separately in one combined unit dosage form or in two separate unit dosage forms. The unit dosage form may also be a fixed combination.

The pharmaceutical compositions for separate administration of the mTOR inhibitor a) and co-agent b) or for the administration in a fixed combination, i.e. a single galenical composition comprising at least two combination partners a) and b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

Suitable pharmaceutical compositions contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as tablets, capsules or suppositories, or ampoules. If not indicated otherwise, these are prepared in a manner known per se, for example by means of conventional mixing, granulating, dissolving or lyophilizing processes. It will be appreciated that the unit content of a combination partner contained in an individual dose of each dosage form need not in itself constitute an effective amount since the necessary effective amount can be reached by administration of a plurality of dosage units.

For example, the method of delay of progression or treatment of arthritis, rheumatic arthritis or disorders associated therewith according to the invention may comprise (i) administration of the mTOR inhibitor a) in free or pharmaceutically acceptable salt form and (ii) administration of a co-agent b) in free or pharmaceutically acceptable salt form, simultaneously or sequentially in any order, in jointly therapeutically effective amounts, preferably in synergistically effective amounts, e.g. in daily or intermittently dosages, e.g. corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. A physician, clinician or veterinarian of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites, particularly when co-agent b) is a small molecule.

Daily dosages for the mTOR inhibitor a) will, of course, vary depending on a variety of factors, for example the compound chosen, the particular condition to be treated and the desired effect. In general, however, satisfactory results are achieved on administration of agent a) at daily dosage rates of the order of ca. 0.01 to 5 mg/kg per day, particularly 0.5 to 5 mg/kg per day, as a single dose or in divided doses. A preferred daily dosage range is about from 0.1 to 30 mg as a single dose or in divided doses. The mTOR inhibitor a), e.g. Compound A, may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.05 to 15 mg active ingredient, usually 0.25 to 10 mg, e.g. Compound A, together with one or more pharmaceutically acceptable diluents or carriers therefor.

The co-agent b) may be administered in a dosage range as known in the art, e.g. at the lower known dosage ranges.

Methotrexate may be administered to a human in the following dosage ranges: 0.1 mg/kg daily every 2 or 3 days p.o.

Infliximab may be administered to a human in the following dosage ranges: 3 mg/kg iv intermittently, e.g. weeks 1,2 and 6 and then every 8^{th} week.

Etanercept may be administered to a human in the following dosage ranges: 2×25 mg/week.

Celecoxib may be administered to a human in the following dosage ranges: 200-400 mg/day p.o.

Rapamycin or derivatives thereof are well tolerated at dosages required for use in accordance with the present invention. For example, the NTEL for Compound A in a 4-week toxicity study is 0.5 mg/kg/day in rats and 1.5 mg/kg/day in monkeys.

1. A pharmaceutical combination comprising:
a) mTOR inhibitor, and
b) at least one co-agent shown to have clinical activity against arthritis or rheumatic arthritis.

2-7. (canceled)

8. A pharmaceutical composition for reducing or inhibiting macrophages or synovial fibroblasts proliferation, for preventing, alleviating or treating pain or for preventing, alleviating or treating pyresis, comprising a mTOR inhibitor together with one or more pharmaceutically acceptable diluents or carriers therefore.

9. A method for treating arthritis, rheumatic arthritis or disorders associated therewith in a subject in need thereof comprising co-administration to said subject of a therapeutically effective amount of a mTOR inhibitor and at least one co-agent shown to have clinical activity against arthritis or rheumatic arthritis.

10. The combination of claims 1, wherein the mTOR inhibitor is 40-0-(2-hydroxyethyl)-rapamycin.

11. The composition of claim 8, wherein the mTOR inhibitor is 40-0-(2-hydroxyethyl)-rapamycin.

12. The method of claim 9, wherein the mTOR inhibitor is 40-0-(2-hydroxyethyl)-rapamycin.

13. The composition of claim 8 in combination with at least one co-agent shown to have clinical activity against arthritis or rheumatic arthritis.

14. The method of claim 9 wherein the mTOR inhibitor is used for slowing the progression, in a subject having moderate to severe rheumatoid arthritis.

15. A method for reducing or inhibiting macrophages or synovial fibroblasts proliferation in a subject comprising administering to said subject a therapeutically effective amount of a mTOR inhibitor and optionally co-administered in combination with a therapeutically effective amount of at least one co-agent show to have clinical activity against arthritis or rheumatic arthritis.

16. The method of claim 15 wherein the mTOR inhibitor is used for reducing or inhibiting bulk formation of invasive fibrous pannus in a subject.

17. A method for preventing, alleviating or treating pain comprising administering to said subject a therapeutically effective amount of a mTOR inhibitor and optionally co-administered in combination with a therapeutically effective amount of at least one co-agent shown to have clinical activity against arthritis or rheumatic arthritis.

18. A method for preventing, alleviating or treating pyresis comprising administering to said subject a therapeutically effective amount of a mTOR inhibitor and optionally co-administered in combination with a therapeutically effective amount of at least one co-agent shown to have clinical activity against arthritis or rheumatic arthritis.
